(19) United States
(12) Patent Application Publication
(10) Pub. No.: US 2010/0121197 A1
(43) Pub. Date: May 13, 2010

(54) ULTRASONIC HORN WITH ENLARGED DISTAL END PORTION

(75) Inventors: Hidefumi OTA, Tokyo (JP); Kazuyuki SUZUKI, Tokyo (JP)

Correspondence Address: CANTOR COLBURN, LLP, 20 Church Street, 22nd Floor, Hartford, CT, 06103, US

(73) Assignee: Miwatec Co., Ltd., Tokyo (JP)

(21) Appl. No.: 12/429,495

(22) Filed: Apr. 24, 2009

(30) Foreign Application Priority Data
Apr. 25, 2008 (JP) JP2008-115286

(51) Int. Cl. A61B 8/14

(52) U.S. Cl. 600/462

(57) ABSTRACT

An ultrasonic horn includes a main body portion and a distal end structure formed at a distal end of the main body portion, the ultrasonic horn being adapted to be used to cut living tissue. The largest outer diameter of a portion of the ultrasonic horn is in the distal end structure, the portion being inserted into the living tissue when the living tissue is cut.

The present application is based on, and claims priority from, J.P. Application No. 2008-115286, filed on Apr. 25, 2008, the disclosure of which is hereby incorporated by reference herein in its entirety.

### BACKGROUND OF THE INVENTION

1. Field of the Invention

The present invention relates to an ultrasonic horn and an ultrasonic hand piece, and in particular, relates to an ultrasonic horn and an ultrasonic hand piece suitable for cutting living bone tissue.

2. Description of the Related Art

In a conventional orthopedic operation of a spine or a cervical spine, a treatment including; (a) fixing a plurality of pedicle screws to centrums and (b) coupling the pedicle screws to a plate in order to fix the centrums together has been commonly used. In order to fix a pedicle screw to a centrum, a doctor normally cuts and removes the vertebral arch from the centrum, and then uses a drill to form a guide hole in the cancellous bone of the pedicle into which a pedicle screw is to be screwed. The guide hole typically has a diameter ranging between 3 and 4 mm and a length of about 13 mm.

The cancellous bone is exposed over a relatively large area in the part of the centrum where the vertebral arch is removed. The cancellous bone is connected to the interior of the centrum through a narrow area sandwiched between cortical bones. Since a radicle of a nerve is present adjacent to the narrow area and is very close to the cortical bone, breaking through the cortical bone with the drill may lead to damage on the surrounding nerves or vessels. Furthermore, if the pedicle screw is not fixed in an appropriate direction, then adjacent radicles may be compressed by the force that is exerted on the pedicle screw when the pedicle screw is coupled to the plate. Accordingly, it is necessary that the guide hole be formed such that the pedicle screw can be screwed straight toward the centrum through the region between the cortical bones.

Normally, when a guide hole is formed by means of a drill, a doctor manipulates the drill while checking the location at which the guide hole is formed by means of X-ray pictures. However, appropriately forming the guide hole relying on the X-ray pictures, which are two-dimensional images, requires a significantly high level of skill. Further, taking an X-ray picture requires suspension of the operation. Thus, it is desirable to form a guide hole without taking X-ray pictures in view of the need of rapid treatment and relieving the situation of the patient.

Under these circumstances and as a result of elaborate work, the present inventors found that the guide hole can be formed in the cancellous bone of the pedicle safely and easily by using an ultrasonic operative instrument (an ultrasonic hand piece). Irrespective of the lower cutting efficiency as compared to a drill, the ultrasonic operative instrument still offers easy cutting of the cancellous bone, in which the guide hole is formed, while it can not easily cut the cortical bone that is harder than the cancellous bone. This enables a doctor to clearly feel with his hand the difference between the cancellous bone and the cortical bone when an ultrasonic operative instrument reaches the cortical bone from the cancellous bone. Thus, a doctor can safely and easily form a guide hole without checking X-ray pictures by cutting a cancellous bone and by relying on his hand to feel the difference between cancellous and cortical bone.

A known ultrasonic operative instrument described above is disclosed in Japanese Patent Laid-Open No. 2005-152098.

An ultrasonic operative instrument includes an ultrasonic vibration mechanism for generating ultrasonic vibration and a horn that utilizes the vibration transmitted from the ultrasonic vibration mechanism in order to perform cutting at a distal end structure thereof. The distal end structure of the horn is generally formed in a straight tube having a constant outer diameter. However, a distal end structure having a constant outer diameter increases the side area of the horn that comes into contact with living tissue as cutting progresses and as the distal end structure is gradually inserted into the living tissue, such as a bone. An increase in the area of a horn that comes into contact with the living tissue produces higher friction between the horn and the living tissue, leading to a reduction in living tissue cutting efficiency and to generation of heat at the interface between the living tissue and the horn.

### SUMMARY OF THE INVENTION

An object of the present invention is to provide an ultrasonic horn and an ultrasonic hand piece, as well as a treatment method using the same, that can prevent a reduction in cutting efficiency that may occur as cutting progresses.

To this end, an ultrasonic horn according to the present invention comprises a main body portion and a distal end structure formed at a distal end of the main body portion, the ultrasonic horn being adapted to be used to cut living tissue. The largest outer diameter of a portion of the ultrasonic horn is in the distal end structure, the portion being inserted into the living tissue when the living tissue is cut.

According to one embodiment of the present invention, an ultrasonic hand piece comprises: an ultrasonic vibration mechanism for generating ultrasonic vibration, an ultrasonic horn described above, the ultrasonic horn being coupled to the ultrasonic vibration mechanism, and an outer cylindrical portion covering the ultrasonic vibration mechanism and a part of the ultrasonic horn.

According to another embodiment of the present invention, a method for cutting living tissue to create a hole in the living tissue comprises inserting an ultrasonic horn into the living tissue, the ultrasonic horn including a main body portion and a distal end structure formed at a distal end of the main body portion, wherein a largest outer diameter of a portion of the ultrasonic horn is in the distal end structure, the portion being inserted into the living tissue when the living tissue is cut.

The present invention can provide an ultrasonic horn and an ultrasonic hand piece, as well as a treatment method using the same, that can prevent a reduction in cutting efficiency that may occur as cutting progresses.

The above and other objects, features and advantages of the present invention will become apparent from the following description with reference to the accompanying drawings which illustrate examples of the present invention.

### BRIEF DESCRIPTION OF THE DRAWINGS

FIG. 1 illustrates an ultrasonic hand piece according to an embodiment of the present invention;

FIG. 2 is a sectional view showing the outer cylindrical portion and the internal structure of the horn of the ultrasonic hand piece;

FIG. 3 illustrates in details a vibration converting mechanism;

FIG. 4 illustrates the operation of the distal end of the horn;

FIG. 5 is a partial sectional view of a horn according to an embodiment of the present invention;

FIG. 6 illustrates how living tissue is cut with the horn;

FIG. 7 illustrates how the distal end structure of the horn that cuts the cancellous bone is guided to the region between the cortical bones;

FIGS. 8A and 8B illustrate a chamfer formed on the distal end structure of the horn;

FIG. 9 is a sectional view showing a first variation of the horn shown in FIG. 5;

FIG. 10 illustrates how bone chips are sucked in from the suction port of the horn, the bone chips being produced by the horn that cuts the born;

FIGS. 11A and 11B are sectional views showing further variations of the horn shown in FIG. 9;

FIG. 12 illustrates a second variation of the horn shown in FIG. 5; and

FIGS. 13A and 13B are partial sectional views showing horns having other external shapes.

### DETAILED DESCRIPTION OF THE INVENTION

Embodiments of the present invention will now be described with reference to the drawings.

### (Ultrasonic Hand Piece)

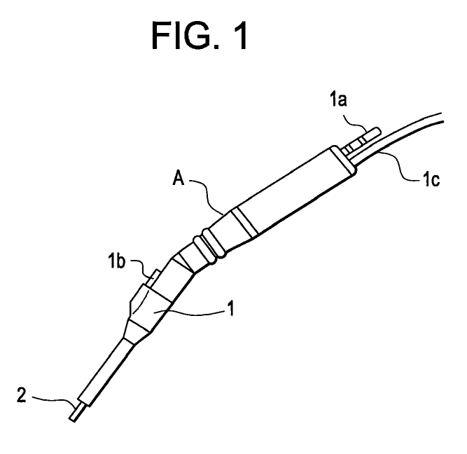

First, an ultrasonic hand piece according to an embodiment of the present invention will be described with reference to FIGS. 1 to 4.

FIG. 1 illustrates an ultrasonic hand piece according to an embodiment of the present invention.

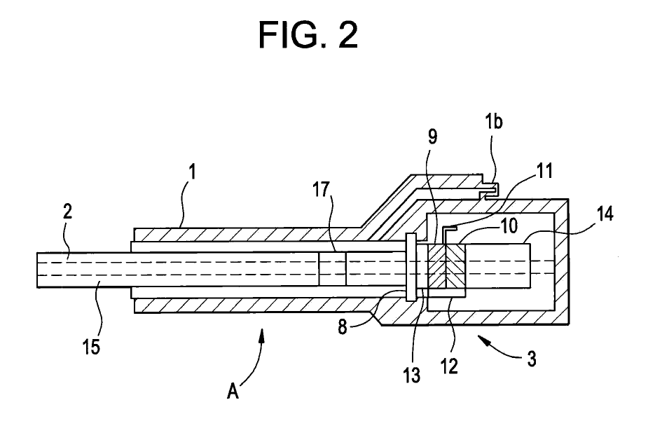

Referring to FIG. 1, outer cylindrical portion 1 houses ultrasonic vibration mechanism 3 (see FIG. 2) that includes a vibrator, such as a magnetostrictive type or an electrostrictive type, that outputs ultrasonic waves of a predetermined frequency. Horn 2 is inserted into outer cylindrical portion 1 at one end thereof. Horn 2 is adapted to cut hard living tissue, such as a bone, with a distal end structure thereof by the aid of vibration that is transmitted from ultrasonic vibration mechanism 3. Irrigation liquid, pieces of living tissue cut into fragments, etc. are sucked out to a tube, not shown, via connection 1a. Irrigation liquid is injected from a tube, not shown, via connection 1b. Ultrasonic vibration mechanism 3 is supplied with high-frequency electric energy via Cable 1c.

Vibration transmitted from ultrasonic vibration mechanism 3 causes horn 2 to vibrate at a predetermined frequency in an axial direction thereof. Horn 2 cuts a desired portion of hard living tissue, such as a bone, at the distal end thereof that is in touch with the hard living tissue.

FIG. 2 is a sectional view showing the outer cylindrical portion of the ultrasonic hand piece and the internal structure of the horn shown in FIG. 1.

Outer cylindrical portion 1 is configured to cover horn 2 and ultrasonic vibration mechanism 3 that is coupled to the proximal end of horn 2. Ultrasonic vibration mechanism 3 includes flange 8, piezo elements 9, 10, electrodes 11, 12, front plate 13 and back plate 14. Piezo elements 9, 10, which are sandwiched between front plate 13 and back plate 14, generate vibration in the axial direction (in the traversing direction of FIG. 2), in response to high-frequency power that is supplied to electrodes 11, 12 via cable 1c shown in FIG. 1. Front plate 13 is integrated with flange 8. Back plate 14 is supported by a support member, not shown, provided at the side opposite to piezo element 10. Piezo elements 9, 10, front plate 13 and back plate 14 are supported within outer cylindrical portion 1 by means of flange 8 and the support member so that they are restricted in the direction of vibration.

These elements are integrated by means of screws provided at the junction of the elements adjacent to each other.

The ultrasonic operative instrument utilizes ultrasonic vibration to crush, emulsify and suck in human living tissue in order to selectively remove the damaged portion. The distal end structure of horn 2 projecting from outer cylindrical portion 1 generates a large quantity of heat because it is the portion that comes into actual contact with the human portion to be cut and vibrates at a large amplitude (displacement). Consequently, joint 1b is provided on the outer periphery of outer cylindrical portion 1. Joint 1b is used to inject irrigation liquid in order to cool the distal end structure and to promote suction of removed objects.

Suction through-hole 15 is provided to extend through horn 2, front plate 13, piezo elements 9, 10, back plate 14 and outer cylindrical portion 1, which are configured as described above, along the central line of these elements. Crushed and emulsified living tissue is sucked in by an external suction pump via suction through-hole 15 and joint 1a shown in FIG. 1. These elements described above are formed substantially rotationally symmetric with respect to suction through-hole 15.

Furthermore, vibration converting mechanism 17 is provided on horn 2. Vibration converting mechanism 17 is the same as that disclosed in JPA 2005-152098, the disclosure of which is hereby incorporated by reference herein in its entirety.

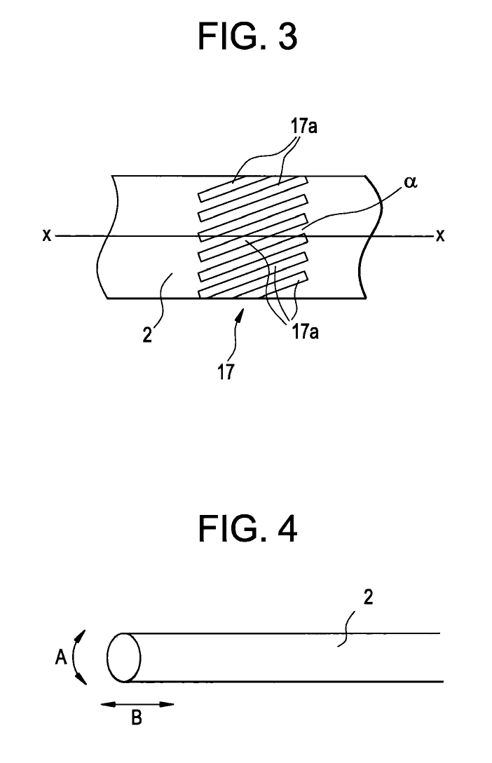

FIG. 3 illustrates in detail vibration converting mechanism 17. As shown in FIG. 3, vibration converting mechanism 17 is comprised of a plurality of groove portions 17a that are wound around the side surface of horn 2.

Groove portions 17a are engraved on horn 2 in parallel with each other at a predetermined interval. Each groove portion 17a is inclined on the side surface at predetermined angle α, which is set to be more than 0 degree and less than 90 degrees, with respect to central axis X-X of horn 2.

Groove portion 17a is formed in a rectangle having a width of 0.5 to 5 mm, a length of 3 to 30 mm and a depth of at least 0.5 mm.

It should be noted that the location of the groove portions serving as vibration converting mechanism 17 is not limited to the side surface of horn 2. The groove portions may be formed at a location between the distal end of horn 2 and the electrostrictive element of ultrasonic vibration mechanism 3, wherein the location includes the outer side surfaces of horn 2, ultrasonic vibration mechanism 3 and any members interposed between horn 2 and ultrasonic vibration mechanism 3.

FIG. 4 illustrates the operation of the distal end of horn 2. Vibration converting mechanism 17 converts longitudinal vibration to a combination of longitudinal and torsional vibrations, causing the distal end of horn 2 to perform high-speed pivotal movement around the central axis in the direction of arrow A (torsional vibration), as well as high-speed reciprocating movement along the central axis in the direction of arrow B (longitudinal vibration).

The mechanism of vibration conversion performed by groove portions 17a can, at present, be explained as follows. As shown in FIG. 3, groove portions 17a are repeatedly deformed by longitudinal vibration. During deformation, part of the longitudinal vibrational component is considered to be converted into the torsional vibrational component.

The above-described configuration enables a synthesized movement comprised of a high-speed pivotal movement and a high-speed reciprocating movement, and significantly improves not only the efficiency in cutting living tissue but also the sharpness in a cutting action, i.e., cutting quality. Thus, it is possible to cut a damaged site undisturbed without crushing adjacent living tissue.

In use, the distal-end tip of horn 2 is pressed against the site to be cut in order to crush and emulsify the living tissue in the site. The irrigation liquid injected via joint 1b cools horn 2 when it flows between cylindrical portion 1 and horn 2, and, after being discharged from cylindrical portion 1, is sucked out to the outside via suction through-hole 15 together with pieces of the living tissue cut into fragments.

### (Ultrasonic Horn)

An embodiment of the ultrasonic horn according to the present invention will now be described.

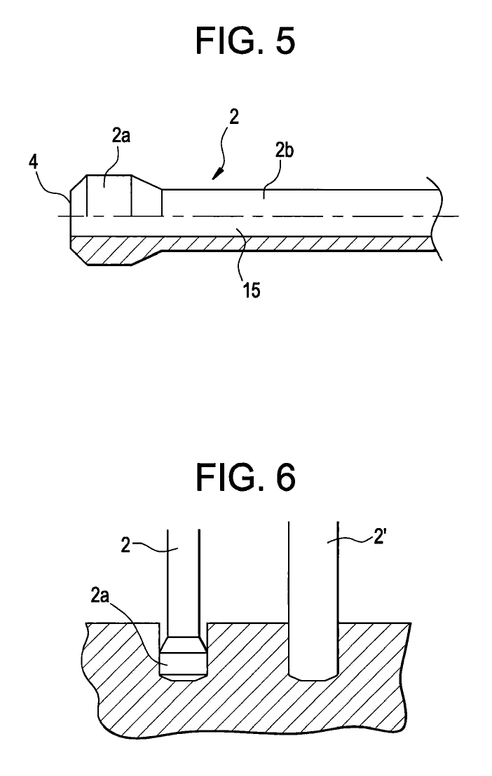

FIG. 5 is a partial sectional view showing an ultrasonic horn according to an embodiment of the present invention. Horn 2 shown in FIG. 5 includes main body portion 2b and distal end structure 2a formed at distal end 4 of main body portion 2b. At least regarding the part of horn 2 which is inserted into living tissue, such as a bone, during cutting, distal end structure 2a has a larger outer diameter than the other portions. Suction through-hole 15 is formed within horn 2 and extends through horn 2 from distal end 4 to the proximal end of horn 2, as with the configuration described above with reference to FIG. 2.

Horn 2 according to the present embodiment is 110 mm in total length; main body portion 2b is 2.5 mm in outer diameter; and suction through-hole 15 is 2.0 mm in diameter. Distal end portion 2a is 2.8 mm in diameter and 3.0 mm in length. However, these dimensions are mere examples of horn 2 and do not limit the horn according to the present invention.

FIG. 6 illustrates how living tissue is cut with the horn.

Horn 2 according to the present embodiment shown in the left part of FIG. 6, which has distal end structure 2a formed thicker than the other portions, keeps only distal end structure 2a of horn 2 in contact with the surrounding living tissue even when cutting of the living tissue progresses to some extent. Thus, the area of the horn that is in contact with the surrounding living tissue is kept constant regardless of the depth to which the living tissue has been cut, and the magnitude of friction between horn 2 and the surrounding living tissue is not increased as the process of cutting of living tissue progresses. Consequently, a reduction in the efficiency of horn 2 in cutting the living tissue can be prevented, and heat generated in the contact portion between the living tissue and horn 2 can be reduced.

On the other hand, in the case of horn 2′ having a constant outer diameter, as shown in the right part of FIG. 6, the area of horn 2′ that is in contact with the living tissue is increased, as described above, as cutting progresses and as the distal end structure is gradually inserted into the living tissue. This causes increased friction between horn 2′ and the living tissue, thereby reducing efficiency of horn 2′ in cutting the living tissue, as well as increasing heat generated in the contact portion between the living tissue and horn 2′.

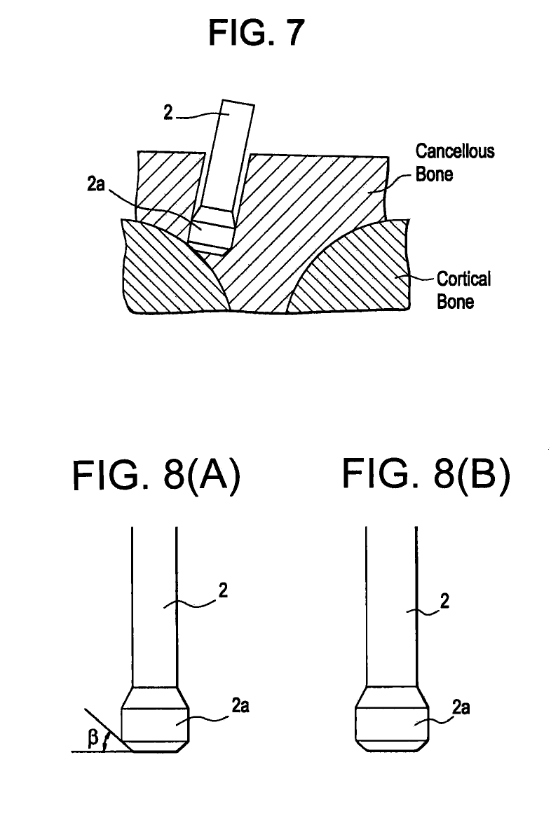

FIG. 7 illustrates how the distal end structure of the horn that is cutting the cancellous bone is guided into the region between the cortical bones.

The distal edge of distal end structure 2a of horn 2 according to the present embodiment is chamfered. Thus, even if distal end structure 2a of horn 2 is moved away from the region between the cortical bones in the cutting process and distal end structure 2a comes into contact with the cortical bone, damage on the cortical bone can be avoided because of the dull distal edge of distal end structure 2a. Moreover, as described above, when distal end structure 2a of horn 2 reaches the cortical bone from the cancellous bone, a doctor can recognize this with his hand. Thus, the chamfered portion can be slid along the cortical bone in order to modify the cutting direction, as shown by an arrow in FIG. 7, and a drill hole can be correctly formed in the direction of the region between the cortical bones.

FIGS. 8A and 8B illustrate the chamfered portions formed at the distal end structure of the horn. Distal end portion 2a of horn 2 may be linearly chamfered, as shown in FIG. 8A, or may be curvilinearly chamfered, as shown in FIG. 8B. In the case of the linearly chamfered distal end structure shown in FIG. 8A, the angle β between the distal end surface of the horn and the chamfer forming surface may be selected from any value range that satisfies the relationship 0<β<90°, for example, 20≦β≦80°. In the case of the curvilinearly chamfered distal end structure shown in FIG. 8B, the curved portion may have any curvature.

### [Variation of the Ultrasonic Horn]

### <First Variation>

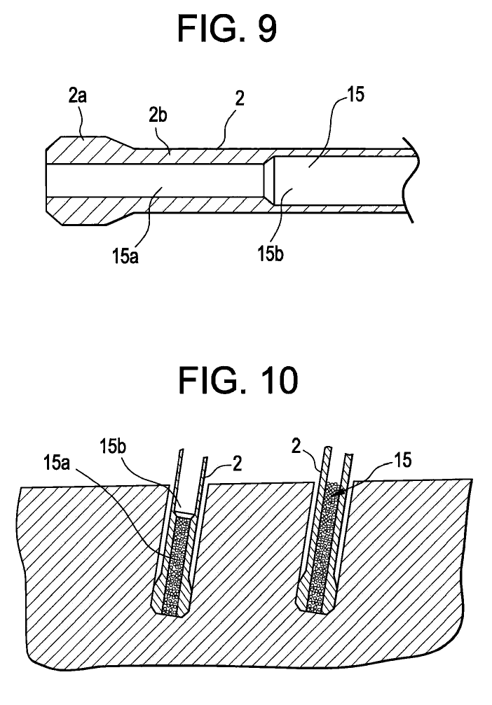

A first variation of the horn shown in FIG. 5 will now be described with reference to FIGS. 9, 10.

FIG. 9 is a sectional view showing the first variation of the horn shown in FIG. 5. Suction through-hole 15 formed inside horn 2, as shown in FIG. 9, includes first diameter portion 15a, with one end thereof open at the distal end of horn 2, and second diameter portion 15b having a larger diameter than first diameter portion 15a and communicating with the other end of first diameter portion 15a.

FIG. 10 illustrates how bone chips are sucked in from the suction port of the horn, the bone chips being produced by the horn that cuts the born. The horn shown in the left part of FIG. 10 corresponds to that shown in FIG. 9, and the horn shown in the right of FIG. 10 corresponds to that shown in FIG. 5.

According to the present embodiment, the area of the horn that is in contact with the living tissue (actual crushing area) is reduced, and accordingly, there is an improvement in the efficiency in cutting living tissue because distal end structure 2a has a larger diameter than the other portions. Therefore, a larger amount of bone fragments to be sucked out through suction through-hole 15 per unit time is discharged as compared to a conventional horn. In a normal condition, it is possible for the configuration having suction through-hole 15 with a constant diameter to successfully suck in bone fragments or the like, as shown in the right part of FIG. 10. However, suction through-hole 15 of this configuration may be clogged with the bone fragments or the like in the distal region of suction through-hole 15. The bone fragments or the like that clog suction through-hole 15 may prevent horn 2 from performing ultrasonic vibration and may reduce the cutting efficiency. Furthermore, the bone fragments or the like generated by cutting may stay in the hole and may prevent further cutting action.

In contrast, suction through-hole 15 of horn 2 shown in the left part of FIGS. 9, 10 includes first diameter portion 15a and second diameter portion 15b, as described above. Thus, when the bone chips or the like sucked in via first diameter portion 15a reach second diameter portion 15b, the bone chips in a massive state loosen into particles, and thus clogging in suction through-hole 15 can be prevented. Accordingly, a reduction in cutting efficiency due to the clogging of the bone chips or the like in suction through-hole 15 can be prevented. Similarly, bad effect on cutting that may be caused by the bone fragments or the like that are generated by cutting and that stay in the hole can be prevented.

Furthermore, because of the expanding and contracting movements of horn 2 during an operation, horn 2 is subjected to stress because of these movements. This stress may cause metal fatigue in horn 2, which may result in the failure of horn 2. In particular, distal end structure 2a of horn 2 tends to be more easily damaged because of the stress that occurs during the operation and additionally because of the pressing force that horn 2 exerts against the living tissue during cutting. However, horn 2 shown in the left part of FIGS. 9, 10 is provided with first diameter portion 15a having a smaller inner diameter than the other portions on the side of the distal end. Thus, distal end structure 2a of horn 2 is less apt to be damaged because of the larger thickness and the improved strength of distal end structure 2a of horn 2.

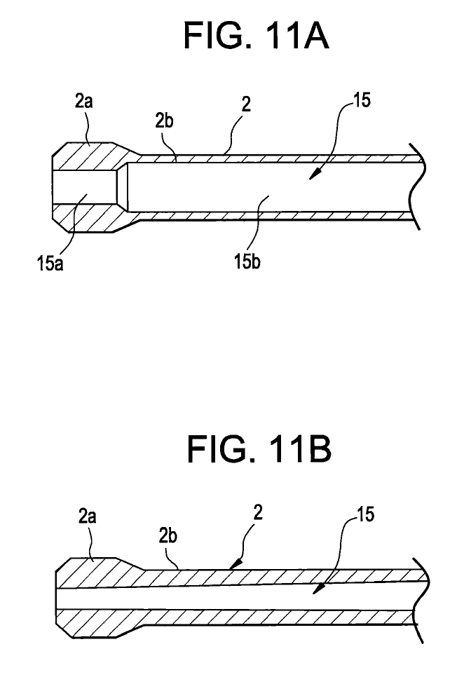

FIGS. 11A and 11B are sectional views showing further variations of the horn shown in FIG. 9.

First diameter portion 15a preferably has a reduced length in order to prevent suction through-hole 15 from being clogged with the bone chips or the like. In the example shown in FIG. 11A, first diameter portion 15a is formed only in the region of horn 2 in which distal end structure 2a is formed and has almost the same length as distal end structure 2a. Consequently, first diameter portion 15a shown in FIG. 11A has a shorter length than first diameter portion 15a shown in FIG. 9. According to the configuration shown in FIG. 11A, the bone chips or the like are fed to second diameter portion 15b before they clog first diameter portion 15a, and suction through-hole 15 can be substantially prevented from being clogged with the bone chips or the like. The increased thickness of distal end structure 2a of horn 2, and accordingly, increased strength thereof, can reduce the possibility that distal end structure 2a will be damaged. Moreover, the configuration provided with first diameter portion 15a having a shorter length than distal end structure 2a can further reduce the possibility that suction through-hole 15 will be clogged with the bone chips or the like.

Moreover, as shown in FIG. 11B, suction through-hole 15 may be tapered such that the diameter of suction through-hole 15 is gradually increased from the distal end to the proximal end of main body portion 2b. This configuration can also prevent suction through-hole 15 from being clogged with the bone chips or the like. Furthermore, the increased thickness of distal end structure 2a of horn 2 and increased strength thereof can reduce the possibility that distal end structure 2a will be damaged.

### <Second Variation>

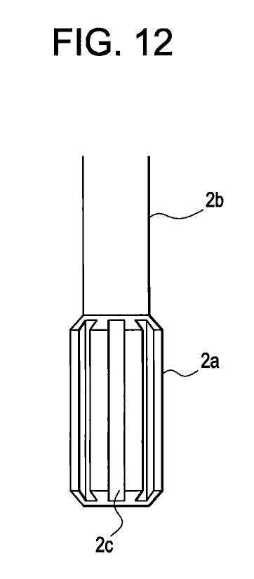

FIG. 12 illustrates a second variation of the horn shown in FIG. 5.

Horn 2 shown in FIG. 12 includes a plurality of grooves 2c that are formed on the side surface of distal end structure 2a and that extend in the longitudinal direction of horn 2. As described above with reference to FIG. 4, distal end structure 2a of horn 2 vibrates torsionally around the central axis and longitudinally along the central axis. Thus, horn 2 shown in FIG. 12 cuts the living bone tissue or the like with the edges of grooves 2c due to torsional vibration. Consequently, the present variation further improves the living tissue cutting efficiency because the side surface of distal end structure 2a contributes to cutting. In addition, grooves 2c allow the bone chips or the like generated during cutting to be removed through grooves 2c, reducing the possibility that cutting of the living tissue will be hindered by the bone fragments or the like that remain in the hole that has been formed.

### (Other Variations)

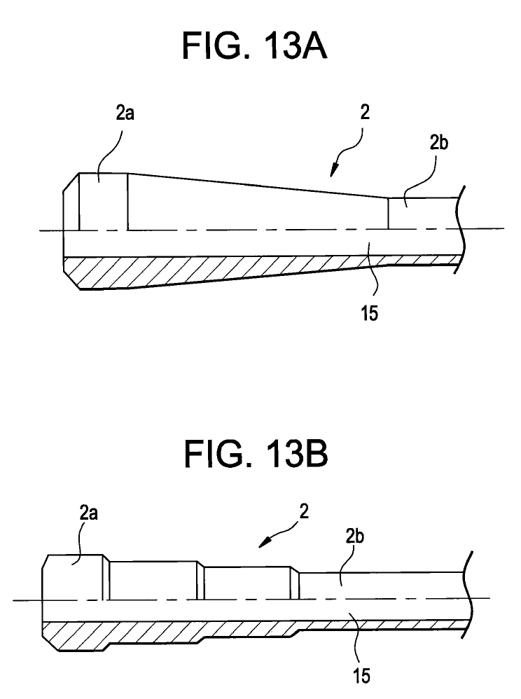

With reference to FIGS. 5, 9, etc., horn 2 that includes distal end structure 2a and main body portion 2b having a smaller outer diameter than distal end structure 2a, i.e., horn 2 having two outer diameters, have been described. However, the external shape of horn 2 is not limited to these embodiments. For example, at least the portion of horn 2 which is inserted into the living tissue, such as a bone during cutting, may have a tapered external shape whose outer diameter is gradually decreased from distal end structure 2a toward main body portion 2b, as shown in FIG. 13A. Alternatively, at least the portion of horn 2 which is inserted into the living tissue, such as a bone during cutting, may have a multi-stepped external shape whose outer diameter is decreased stepwise from distal end structure 2a toward main body portion 2b, as shown in FIG. 13B.

The present invention has been described in conjunction with the embodiments and the variations thereof. However, the present invention is not limited to the embodiments and the variations. Furthermore, the configurations according to the embodiments and variations may be combined together where possible. It also should be understood that various changes and modifications may be made without departing from the spirit or scope of the appended claims.

What is claimed is:

1. An ultrasonic horn comprising a main body portion and a distal end structure formed at a distal end of the main body portion, the ultrasonic horn being adapted to be used to cut living tissue,
wherein a largest outer diameter of a portion of the ultrasonic horn is in the distal end structure, the portion being inserted into the living tissue when the living tissue is cut.

2. The ultrasonic horn according to claim 1, wherein a plurality of grooves is formed on a side surface of the distal end structure, the grooves extending in a longitudinal direction of the ultrasonic horn.

3. The ultrasonic horn according to claim 1, wherein a distal edge of the distal end structure is chamfered.

4. The ultrasonic horn according to claim 1, wherein a through-hole is formed in the main body portion, the through-hole extending through the main body portion from the distal end thereof to a proximal end thereof.

5. The ultrasonic horn according to claim 4, wherein the through-hole includes a first diameter portion, one end thereof being open at the distal end of the horn, and a second diameter portion having a larger diameter than the first diameter portion and communicating with the other end of the first diameter portion.

6. The ultrasonic horn according to claim 5, wherein the first diameter portion has a length that is substantially the same as or shorter than that of the distal end structure.

7. The ultrasonic horn according to claim 4, wherein the through-hole is tapered such that a diameter of the through-hole is gradually increased from the distal end of the main body portion to a proximal end of the main body portion.

8. An ultrasonic hand piece comprising:
an ultrasonic vibration mechanism for generating ultrasonic vibration,
an ultrasonic horn according to claim 1, the ultrasonic horn being coupled to the ultrasonic vibration mechanism, and
an outer cylindrical portion covering the ultrasonic vibration mechanism and a part of the ultrasonic horn.

9. A method for cutting living tissue to create a hole in the living tissue, comprising inserting an ultrasonic horn into the living tissue, the ultrasonic horn including a main body portion and a distal end structure formed at a distal end of the main body portion, wherein a largest outer diameter of a portion of the ultrasonic horn is in the distal end structure, the portion being inserted into the living tissue when the living tissue is cut.
